(19) United States
(12) Patent Application Publication
(10) Pub. No.: US 2016/0038577 A1
(43) Pub. Date: Feb. 11, 2016

(54) USE OF ANTI-ERBB2 VACCINES IN ASSOCIATION WITH AN ELECTRIC FIELD

(71) Applicant: Indena S.p.A. Con Socio Unico, Milano (IT)

(72) Inventors: Paolo Morazzoni, Milano (IT); Antonella Riva, Milano (IT); Giovanna Petrangolini, Milano (IT); Ruggero Cadossi, Carpi (IT); Mattia Ronchetti, Milano (IT)

(73) Assignee: Indena S.p.A. Con Socio Unico, Milano (IT)

(21) Appl. No.: 14/819,540

(22) Filed: Aug. 6, 2015

(30) Foreign Application Priority Data
Aug. 6, 2014 (IT) TO2014A000638

(51) Int. Cl. A61K 39/00; A61K 41/00; C12N 9/12

(52) U.S. Cl. CPC A61K 39/0011; C12N 9/12; A61K 41/0047; C12Y 207/10001; A61K 2039/53; A61K 2039/54

(57) ABSTRACT

The present invention relates to a plasmid comprising a sequence encoding for a fragment of p185^{neu} chosen in the group consisting of SEQ ID 1, 2, 3, 4, 5, 6 carried by applying a pulsating voltage having an intensity comprised in the range from 100 to 200 V, through a needle electrode for use in the preventive or therapeutic treatment of subjects at risk of developing p185^{neu}-positive tumors, or of patients with p185^{neu}-positive primary tumors, metastasis or relapses.

The present invention relates to the use of anti-ErbB-2 vaccines in therapy following administration by means of electroporation.

### BACKGROUND OF THE INVENTION

Progression of carcinogenesis through different stages requires the activation of specific genes and metabolic pathways and can be blocked by interfering selectively with these.

The characterization of genes differentially expressed during carcinogenesis leads to the identification of metabolic pathways and of preferred molecules against which to trigger an immune response.

Oncoantigens expressed at the pre-neoplastic stage and then superexpressed in the resulting tumor can be effective immunological targets and therefore offer an opportunity to vaccinate the host against the mutated cells as soon as they appear, even after removal of the tumor.

If the progression or recurrence of the tumor can be blocked through an early immune response against these oncoantigens, inhibition can cause the destruction of transduction pathways useful for proliferation of the tumor and it is therefore possible to lose or greatly reduce the tumorigenic potential.

The receptor tyrosine-kinase ErbB-2 (Neu in rats and Her-2 in humans) is an oncoantigen directly involved in the progression of different types of tumor. Oncoantigens are molecules tolerated by the organism; therefore an immune tolerance towards self-oncoantigens associated with the tumor creates numerous obstacles to the occurrence of an immune response following an effective vaccination against the tumor. A new class of DNA-based vaccines (anti-ErbB-2 plasmids), which express both the rat Neu sequence and the human Her-2 sequence as a single hybrid construct that encodes for the fusion proteins Neu/Her-2, has been described in WO2005/039618. The anti-ErbB-2 vaccines have proved effective in overcoming immune tolerance to ErbB-2 in vitro.

However, the main problem associated with the administration of these vaccines consists in carrying a sufficient amount of nucleic acid into the host cell to be treated, as nucleic acid must be expressed in the transfected cells.

In recent years different approaches have been considered for carrying DNA. One of these (Wolf et al. Science 247, 1465-68, 1990; Davis et al. Proc. Natl. Acad. Sci. USA 93, 7213-18, 1996) consisted in administering a nucleic acid in the form of plasmid into the muscle or into the blood stream in combination with substances that can promote its transfection, such as proteins, liposomes, charged lipids or cationic polymers such as polyethyleneimine which are generally good transfection agents in vitro (Behr et al. Proc. Natl. Acad. Sci. USA 86, 6982-6, 1989; Felgner et al. Proc. Natl. Acad. Sci. USA 84, 7413-7, 1987; Boussif et al. Proc. Natl. Acad. Sci. USA 92, 7297-301, 1995).

With regard to muscle, since Wolff's initial publication showing the capacity of muscle tissue to incorporate DNA injected in free plasmid form (Wolff et al. Science 247, 1465-1468, 1990), numerous authors have attempted to improve this procedure (Manthorpe et al., 1993, Human Gene Ther. 4, 419-431; Wolff et al., 1991, BioTechniques 11, 474-485).

In particular, the procedures tested were as follows:

the use of mechanical solutions to force the entry of DNA into cells by adsorbing the DNA. onto partcles that are then propelled into the tissues (“gene gun”) (Sanders Williams et al., 1991, Proc. Natl. Acad. Sci. USA 88, 2726-2730; Fynan et al., 1993, BioTechniques 11, 474-485). These methods have proved effective in vaccination strategies but they affect only the top layer of the tissue. In the case of the muscle, their use would require a surgical approach in order to allow access to the muscle tissue as the particles are unable to pass through the skin tissue;

the injection of DNA, no longer in free plasmid form but combined with molecules capable of acting as a vehicle to facilitate entry of the complexes into cells. Cationic lipids, which are used in numerous other transfection methods, have to date proved somewhat disappointing, as those tested inhibit transfection (Schwartz et al , 1996, Gene Ther, 3, 405-411). This is also the case for cationic peptides and polymers (Manthorpe et al., 1993, Human Gene Ther. 4, 419-431). The only case in which a favorable combination was obtained appears to be the mixing of DNA with polyvinyl alcohol or polyvinylpyrrolidone. However, the increase in transfection resulting from these combinations only represents a factor of less than 10 compared with the results obtained from DNA in naked form (Mumper et. al., 1996, Pharmaceutical Research 13, 701-709);

pretreatment of he muscle to be treated by injection with solutions that improve the diffusion and/or stability of DNA (Davis et al., 1993, Hum. Gene Ther. 4, 151-159), or to promote entry of the nucleic acid, for example through the induction of cell multiplication or regeneration phenomena. The treatment involves the use of local anesthetics or cardiotoxin, of vasoconstrictors, of endotoxins or other molecules (Manthorpe et al., 1993, Human Gene Ther. 4, 419-431; Danko et al., 1994, Gene Ther. 1, 114-121; Vitadello et al., 1994, Hum. Gene Ther. 5, 11-18). However, these pretreatment protocols are difficult to manage, in particular bupivacaine, which in order to be effective, requires to be injected at doses very close to lethal. The pre-injection. of hyperosmotic sucrose, used to improve diffusion, does not increase transfection levels in the muscle (Davis et al., 1993).

Attempts have also been made to transfect other issues in vivo, such as liver tissue, the respiratory epithelium, the central nervous system and tumoral tissue, either using DNA alone or in combination with synthetic vectors (Cotten and Wagner (1994), Current Opinion in Biotechnology 4, 705; Gao and Huang (1995), Gene Therapy, 2, 710; Ledley (1995), Human Gene Therapy 6, 1129). However, the level of expression of the transgenes has proved to be too low to be able to be used in therapy, although some encouraging results have been obtained for the transfer of a plasmid DNA into the vascular walls (Iires et al. (1996) Human Gene Therapy 7,959 and 989).

Electroporation, or the use of electric fields to increase cell permeability, has also been used recently to promote the transfection of DNA in vitro on cell cultures. This technique has also been tested in vivo to increase the efficacy of antitumoral agents, such as bleomycin.

The main problems linked to the transfer of DNA through electroporation consist in identifying the range of intensity of the electric field that is effective without causing lesions to the treated tissue.

WO2005/039618 illustrates the transfer of DNA through electroporation with square electrodes placed at 3 mm from each other. However, this type of electrode cannot be used to treat humans as it is not suitable to reach the muscle tissue in which the tumor mass is located.

### SUMMARY OF THE INVENTION

The object of the present invention is therefore to provide a reliable system for transferring anti-ErbB-2 vaccines.

This object is achieved by the present invention, as it relates to an anti-ErbB-2 plasmid for use in the preventive or therapeutic treatment of subjects at risk of developing p185^{neu}-positive tumors according to claim 1.

The anti-ErbB-2 plasmids according to the invention are carried after applying in the site of administration a pulsating voltage having an intensity comprised in the range from 100 to 200 V, in particular between 125 and 175 V and more in particular having an intensity of 150 V. The pulses are generated by means of an electrode consisting of a needle matrix made of conductive material, in particular stainless steel for medical applications.

The matrix consists of needles arranged in two parallel rows spaced 4 mm apart, having the same number of needles, this number being between 10 and 6, preferably 8, spaced 3.2 mm from each other. The length of the needles varies from 10 to 40 mm, preferably 20 mm.

The electrode is inserted into the site of administration of the anti-ErbB2 plasmids so as to comprise the site of administration inside the needle matrix.

The pulsating voltage used comprises at least two monopolar electric pulses, each having a time comprised in the range from 20 to 30 milliseconds, preferably 25 milliseconds. The pause between two pulses is comprised in the range from 100 to 500 msec, preferably 200 to 400 msec, more in particular 300 msec. The total time of the treatment can vary from 140 to 560 milliseconds, in particular 350 milliseconds.

The frequency of the pulses varies from 2 to 10 Hz, preferably 3.3 Hz.

The plasmids according to the invention are capable of inducing a strong immune response both of antibody type and mediated by killer and helper T lymphocytes. These plasmids contain a sequence encoding for a fragment of p185^{neu} chosen in the group consisting of SEQ ID 1, 2, 3, 4, 5, 6 (the reference sequences for human and rat p185^{neu} have been filed respectively in GeneBank with Accession number M11730 and X03362).

The p185^{neu} encoding sequences can be inserted in any plasmid vector suitable for human administration. Besides the encoding sequences indicated above, the plasmids according to the invention can contain functional elements for transcription control, in particular a promoter placed upstream of the encoding sequence, preferably the CMV promoter, start and stop transcription sequences, selection markers, such as ampicillin or kanamycin resistance genes, CpG motifs, a site of polyadenylation and optional enhancer or transcription activators.

Transcription control elements should be compatible with use of the vector in humans. In a preferred embodiment, the plasmids of the invention contain at least 4 CpG motifs, preferably at least 8, up to a maximum of 80. The CpG motifs (ATAATCGACGTTCAA) of bacterial origin induce macrophages to secret IL-12, which in turn induces IFN gamma secretion by natural killer cells, thus activating a T helper lymphocyte-mediated response (Chu R.S. et al. 1997, J. Exp. Med., 186: 1623). Therefore, the insertion of CpG motifs in plasmid sequences enhances the immune response induced by the antigen encoded by the plasmid.

The plasmid can also be administered in the form of a pharmaceutical composition formulated with pharmaceutically acceptable vehicles and excipients. The pharmaceutical compositions, in a form suitable for parenteral administration, preferably in the form of injectable solution, are preferably used in DNA vaccination procedures. The principles and methods for DNA vaccination are known to those skilled in the art and are disclosed, for example, in Liu MA 2003; J Int Med 253: 402.

The plasmids according to the invention, suitably formulated, are used in preventive or therapeutic treatment of subjects at risk of developing p185^{neu}-positive tumors, or patients with primary tumors, metastasis or relapses of p185^{neu}-positive tumors. Prevention can be primary, when the tumor is not manifest, or tertiary, in the case of tumor relapse or metastatic process. Tumors that can benefit from treatment with the plasmids of the invention are those chosen from the group of p185^{neu}-positive tumors, in particular neck-head and breast tumors.

According to an embodiment of the invention, the electric field is applied following parenteral administration of the plasmid, for example by intramuscular injection.

Intramuscular administration of the plasmid is particularly preferred due to the nature of the muscle tissue formed by multinucleated cells. Administration in this site therefore increases the possibility of transfection of the plasmid administered and is also easily accessible in therapy.

Application of an electric field in the site of administration of the plasmid according to the present invention enhances transfection of the nucleic acid without damaging the treated tissue.

The electric field must be applied within 5 minutes of administering the plasmid, preferably within 2 minutes.

A further advantage of the use of electroporation in gene therapy consists in the safety provided by local treatment linked to the use of local and targeted electric fields.

The inventors have in particular identified the values of field force and the intensity of the electric field to optimize the transfection of RHuT-IDN6439 plasmids.

It is also possible to modulate and control the effective quantity of transgene expressed by the possibility of modulating the volume of the tissue to be transfected, for example with multiple sites of administration, or the possibility of modulating the shape, the surface and the arrangement of the electrodes.

An additional element of control derives from the possibility of modulating the efficiency of transfection by varying the fjeld intensity, the number, the duration and the frequency of the pulses and, obviously according to the state of the art, the quantity and the volume of nucleic acids to be administered.

In particular, the inventors have found that administration of the plasmid in association with the electric field is more effective if it is administered once a week for four weeks. These administrations are then followed by an interval of 22 weeks without administration, and finally by a new cycle of administrations, once a week for four weeks.

### BRIEF DESCRIPTION OF THE DRAWINGS

The present invention will now be described in detail with reference to the figures of the accompanying drawings, wherein:

FIG. 1 shows a plasmid according to the present invention.

### DETAILED DESCRIPTION OF THE INVENTION

Further characteristics of the present invention will be apparent from the description below of some purely illustrative and non-limiting examples.

### Example 1

### Preparation of the plasmid

The plasmid was prepared using the recombinant DNA technology according to the description in WO2005/039618. The plasmid was constructed on the vector pVAX1 (Invitrogen) with the insertion of a sequence encoding for the transmembrane and extracellular domains of rat Neu and human Her-2.

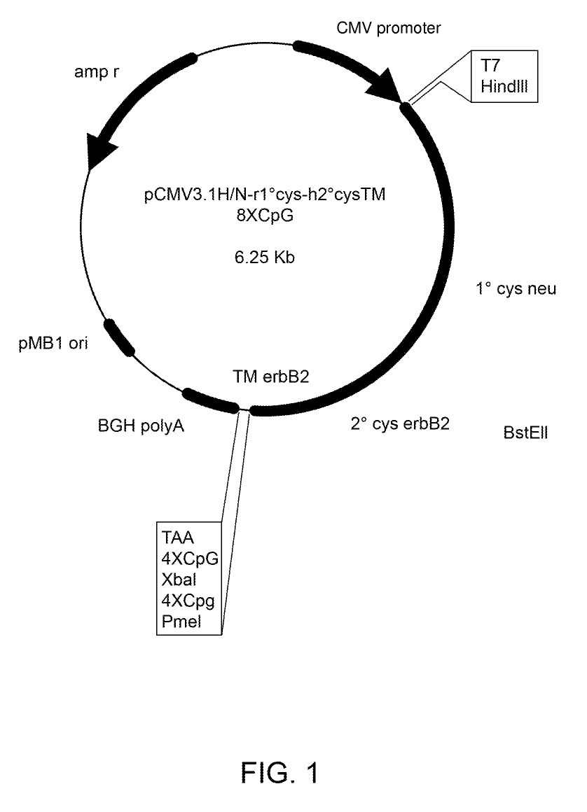

The final plasmid size is 5167 base pairs. The plasmid is shown in FIG. 1. The vector pVAX1 is a plasmid vector specifically designed to develop DNA vaccines. It allows a high number of replications in E.Coli and a high transient protein expression in mammalian cells. The vector contains the following elements:

The CMV Promoter for high expression;

The polyadenylation signal of bovine growth hormone;

The gene for resistance to kanamycin for the selection in E. Coli;

The pUC origin for high replication and growth in E.Coli.

1. Plasmid comprising a sequence encoding a p185^{neu} fragment chosen from the group consisting of SEQ ID N. 1, 2, 3, 4, 5, 6 for use in the preventive or therapeutic treatment of subjects at risk of developing p185^{neu}-positive tumors, or of patients with p185^{neu}-positive primary tumors, metastasis or relapses, characterized in that it is carried by applying a pulsating voltage having an intensity comprised in the range from 100 to 200 V by means of a needle electrode.

2. Plasmid according to claim 1 for the use according to claim 1, characterized in that it further comprises a transcription promoter.

3. Plasmid according to claim 2 for the use according to claim 2, characterized in that said promoter is CMV.

4. Plasmid according to claim 1 for the use according to the claim 1, characterized in that it further comprises at least 4 CpG motifs.

5. Plasmid according to claim 1 for the use according to claim 1, characterized in that said pulsating voltage has an intensity comprised between 125 and 175 V.

6. Plasmid according to the preceding claim for the use according to the preceding claim, characterized in that said pulsating voltage has an intensity of 150 V.

7. Plasmid according to claim 1 for the use according to claim 1, characterized in that said pulsating voltage comprises the repetition of at least two monopolar pulses.

8. Plasmid according to the preceding claim for the use according to the preceding claim, characterized in that the time of each pulse is comprised in the range from 20 to 30 milliseconds.

9. Plasmid according to the preceding claim for the use according to the preceding claim, characterized in that the time of each pulse is 25 milliseconds.

10. Plasmid according to claim 1 for the use according to claim 1, characterized in that said pulsating voltage is administered for a total time comprised in the range from 140 to 560 milliseconds.

11. Plasmid according to the preceding claim for the use according to the preceding claim, characterized in that said electric field is administered for a total time of 350 milliseconds.

12. Plasmid according to claim 1 for the use according to claim 1, characterized in that said electric field is administered with a frequency comprised in the range from 2 to 10 Hz.

13. Plasmid according to the preceding claim for the use according to the preceding claim, characterized in that said electric field is administered at a frequency of 3.3 Hz.

14. Plasmid according to claim 1 for the use according to claim 1, characterized in that said needle electrode has a needle matrix, the length of which varies from 10 to 40 mm.

15. Plasmid according to claim 1 for the use according to claim 1, characterized in that said p185^{neu}-positive tumors are chosen from the group of neck-head and breast tumors.

16. Plasmid according to claim 1 for the use according to claim 1, characterized in that said electric field is applied after the administration of said plasmid.

17. Plasmid according to claim 1 for the use according to claim 1, characterized in that it is administered once a week for four weeks.
